(19) United States
(12) Patent Application Publication
(10) Pub. No.: US 2008/0139991 A1
(43) Pub. Date: Jun. 12, 2008

(54) METHOD OF WOUND DISINFECTING AND TISSUE WELDING

(75) Inventors: Cale Street, Edmonds, WA (US); Nicolas G. Loebel, Bothell, WA (US)

Correspondence Address: DOBRUSIN & THENNISCH PC, 29 W LAWRENCE ST, SUITE 210, PONTIAC, MI, 48342, US

(73) Assignee: Ondine International, Ltd.

(21) Appl. No.: 11/949,439

(22) Filed: Dec. 3, 2007

Related U.S. Application Data
(60) Provisional application No. 60/869,199, filed on Dec. 8, 2006.

(51) Int. Cl. A61B 18/18

(52) U.S. Cl. 604/20; 606/214

(57) ABSTRACT

The present invention includes a method of treating a wound that includes both disinfecting the wound using light and welding the tissue of the wound using light. In a preferred embodiment, the tissue welding occurs substantially immediately after disinfection. The method includes applying a photosensitizer to the wound, illuminating the wound with light, closing the wound, and illuminating the wound a second time. A solder is optionally applied to wound after the first illumination and before the closing of the wound.

### CLAIM OF BENEFIT OF FILING DATE

This application claims the benefit of U.S. Provisional Application Ser. No. 60/869,199 titled: “Method Of Wound Disinfection And Tissue Welding” filed on Dec. 8, 2007.

### FIELD OF THE INVENTION

The present invention relates to methods of treating a wound and more particularly to methods of combining wound disinfection and tissue welding in a single process mediated by light.

### BACKGROUND OF THE INVENTION

Wound disinfection and tissue welding have been foci of attention for the medical community. For wound disinfection, the application of an antimicrobial agent, both locally and systemically, have been used with relative success. Local application of an antimicrobial agent is preferred because any microbial colony is likely to be small and thus more easily treated. One general method of localized disinfection utilizes an energy-mediated free radical reaction to eliminate microbes in the wound. This method generally includes the application of a photosensitizer (also called photoactivators) to the wound followed by the illumination by a laser or other visible light source emitting at a wavelength overlapping the absorbance profile of that photosensitizer resulting in disinfection of the wound through a non-specific antibacterial mechanism.

For tissue welding, traditional bio-adhesives, such as cyanoacrylate glues, have been used to close wounds; however, these adhesives may not be biodegradable and may be toxic. One general method of tissue welding that has gained prominence recently is the use of a solder in the wound that is then illuminated by a light source. The light (e.g. from a laser) may be used to locally heat the solder, distributing thermal energy which reversibly denatures tissue proteins and facilitates the tissue welding reaction. In addition, a photosensitizer incorporated into the solder material may be used to absorb the light and create reactive oxygen species that result in the formation of covalent bonding among the tissue, solder and/or the photosensitizer.

Both light mediated methods fall generally into the classification of photo dynamic therapy (PDT). However, they have been limited to performing one of the two functions but not the other. The inventors have identified a method that performs both wound disinfection and tissue welding in a single procedure.

### SUMMARY OF THE INVENTION

The present invention includes a method of treating a wound that includes both disinfecting the wound using light and welding the tissue of the wound using light. In a preferred embodiment, the tissue welding occurs substantially immediately after disinfection. The method includes applying a photosensitizer composition to the wound, illuminating the wound with light, physically closing the wound edges, and illuminating the wound a second time with a higher energy dose to mediate irreversible closure. A solder is optionally applied to wound after the first illumination and before the closing of the wound.

### DETAILED DESCRIPTION

The present invention includes a method of treating wound including both light mediated disinfection and light mediated tissue welding. Tissue welding occurs substantially immediately after the disinfection to increase the effectiveness of the disinfection, reduce the chances of re-infection, and immediately restore tissue function. The period of time between the two steps is only as long as necessary and may be used to close the wound, apply additional compositions or adjust the illuminating device. The period is preferably less than about 1 minute, less than about 45 seconds, less than about 30 seconds, less than about 20 seconds, or less than about 10 seconds. Substantially immediately means any of these periods of time.

Disinfection includes applying a photosensitizer composition to the wound. The photosensitizer composition includes a photosensitizer that has at least an antimicrobial action when illuminated. Suitable classes of compounds that may be used as antimicrobial photosensitizers include tetrapyrroles or derivatives thereof such as porphyrins, chlorins, bacteriochlorins, phthalocyanines, naphthalocyanines, texaphyrins, verdins, purpurins or pheophorbides, phenothiazines, etc., such as those described in U.S. Pat. Nos. 6,211,335; 6,583,117; and 6,607,522 and U.S. Patent Publication 2003-0180224. Preferred phenothiazines include methylene blue (MB) and those discussed in U.S. Patent publication 2004-0147508. Other preferred antimicrobial photosensitizers include indocyanine green (ICG). Combinations of two or more photosensitizers, such as MB and ICG, are also suitable. The photosensitizer may be present in the photosensitizer composition in amounts between about 0.001 wt % and 1.000 wt %, more preferably between about 0.005 wt % and about 0.5 wt %. While photosensitizers that have other modes of operation (e.g. generation of heat) are contemplated, those that generate reactive oxygen species or free radicals above are preferred.

The photosensitizer composition may also include a solder composition, although this is not necessarily the case. In photosensitizer compositions that do not include a solder composition, the substrate for the tissue welding reaction will be the proteinaceous compounds of the native tissue bordering the wound or weld site. The solder composition preferably comprises a proteinaceous compound, a lipid or both. Suitable proteinaceous compounds include at least one peptide, polypeptide or protein, with preferred proteins including albumin, fibrinogen or gelatin. More preferred proteins include mammalian albumin (e.g. bovine, porcine, human, etc.), with serum albumin being most preferred. Suitably, the photosensitizer and the solder composition form a non-covalent mixture or there is at least one covalent bond that conjugates the photosensitizer to the proteinaceous compound or the lipid, such by a linking moiety. The solder composition may also comprise additional proteinaceous compounds not covalently conjugated to the photosensitizer. The proteinaceous compound covalently conjugated to the photosensitizer may be the same type as the proteinaceous compound that is not covalently conjugated to the photosensitizer, such as albumin for both proteinaceous compounds.

The ratio of total proteinaceous molecules in the composition and the at least one photosensitizer may be from about 100:1 to about 1:100. In certain aspects, the ratio of total proteinaceous molecules in the composition and the at least one photosensitizer is from about 10:1 to about 1:10. In other aspects, the ratio of total proteinaceous molecules in the composition and the at least one photosensitizer is from about 3:1 to about 1:1. In a particular aspect, the ratio of total proteinaceous molecules in the composition and the at least one photosensitizer is about 2:1.

Combinations of any of proteinaceous compounds and lipids are also suitable for use in the solder composition, whether a non-covalent mixture or a covalently bonded molecule, such as a lipoprotein.

Alternately, the solder composition may be separate from the photosensitizer composition and may include its own photosensitizer that is the same or different as the photosensitizer in the photosensitizer compositions. When used alone, the solder composition may include any of the components of the photosensitizer composition discussed below.

The photosensitizer composition may contain a therapeutic agent, which is any chemical, drug, medication, proteinaceous molecule, nucleic acid, lipid, antibody, antigen, hormone, nutritional supplement, cell or any combination thereof that helps ameliorate a condition. Preferred therapeutic agents include those that promote wound healing, have antimicrobial action, have anti-inflammatory action, provide pain relief and/or act as a vasoconstrictor.

The photosensitizer composition may also include a component that acts as a cross-linker that promotes the formation of covalent bonding between and/or among the other components of the composition, such as cross-linking the proteinaceous compound of solder composition. The photosensitizer composition may also include a scaffolding component, consisting of synthetic or biological material (or both), that provides a structure for new cell growth or to fill space within the wound.

The photosensitizer composition may contain a tissue penetration enhancer (e.g. DMSO) that helps increase the absorption by the tissue of one or more of the components of the photosensitizer composition.

The photosensitizer composition may contain carriers, diluents, or other solvents for the photosensitizer or other components of the composition and may be used to adjust the concentration of photosensitizer and/or solder composition in the photosensitizer composition.

The photosensitizer composition may be any suitable phase such as a liquid, gel, paste or solid. Preferably, the compositions has a viscosity low enough to flow into the interstices of the wound while also having a viscosity high enough to maintain the composition within the wound. Further compositions that become liquid after application to the wound are contemplated such as those that melt or go into solution in the wound. Alternately, the composition may gel after application to the wound as a liquid; this would permit the composition to cover the wound effectively, while also maintaining the composition in the wound.

Applying the photosensitizer composition to the wound may be by any suitable technique for discretely placing the composition in the desired location. To a certain extent, the application technique will depend on the viscosity of the composition. Liquid compositions with relatively low viscosities may be sprayed into place, while higher viscosities liquids, solids and/or pastes may be brushed, dabbed, swabbed, or extruded into place. Dry films of the composition may be manually placed in the wound. Preferably the photosensitizer composition is applied in a single portion, although multi-portion composition are also contemplated such as two portions applied at the same time or two or more portion applied sequentially to the wound.

Disinfection also includes the illumination of the photosensitizer composition by an illumination system after it has been placed in the wound. Tissue welding includes the illumination of the closed wound by the illumination system. The illumination system comprises an activation energy source (also called a light source) for applying energy to the wound. For disinfection, the energy is applied to the photosensitizer in the area of the open wound, while for tissue welding the energy is applied to the closed wound where the edges of the wound have been brought together in the presence of the photosensitizer/solder material. Alternately, this may be referred to as closing the flap of the wound.

The light source preferably provides ultraviolet light, infrared light or, most preferable, visible light. The utilized light will have a wavelength matched closely to the absorbance profile of the photosensitizer(s) used. Preferred wavelengths provided by the light source include 660-670 nm and 780-800 nm. In one embodiment, the light source can provide two or more wavelengths at one time or sequentially.

The light source is preferably a laser, although non-coherent light may also be used, such as that produced by an LED or other broadband source. Preferably, the light source is able to deliver light with a power of between about 1 J/cm² and about 100 J/cm², and more preferably between about 5 J/cm² and about 20 J/cm².

The area of illumination provided by the light source is preferably less than the entire area of the wound. In this way, the possibility of damage to the tissue surrounding the wound from the illuminating step can be avoided. In one preferred embodiment, the area of illumination is less than about 1 cm², less than about 0.5 cm², less than about 0.25 cm², or less than about 0.1 cm².

The output of the light source is preferably adjustable so that the operator can modify the wavelength, the power output, the spot-size of illumination, or combinations thereof while carrying out the present method. For example, the wavelength of the laser may be adjusted to activate different photosensitizers in the photosensitizer composition or in the photosensitizer composition and the solder. Alternately, the power of the light source may be increased or decreased after an illumination of the wound for disinfection in order to promote tissue welding. A device with one or more of these attributes reduces the complexity of the wound disinfection and closing procedure thus reducing the operating time.

In addition to the light source, the illumination system may comprise a temperature measurement feature to monitor local temperature at the site of illumination so that over heating of the tissue in and around the wound may be avoided. This feature may be designed as an open loop whereby the temperature measurements are displayed to the user to aid in manual adjustment of the energy source, or as a closed loop whereby the feedback from the temperature measurements actively controls the energy delivery to maintain a specific temperature range at the treatment site. Suitable temperature monitoring devices may comprise an IR device, a fiberoptic device or a thermocouple.

After disinfection and before tissue welding, the wound is closed. As discussed above, the period between disinfection and before tissue welding is preferably less than about 1 minute. Closing the wound is the result of mechanically or physically bringing the edges of the tissue around the wound together so that the edges are adjacent to each other or otherwise placing a flap of the tissue to substantially close the wound by covering it. The disinfection and tissue welding procedure is suitable for use in vertebrate and invertebrate animals with use in mammals being preferred. Use in the veterinary setting is contemplated, but with use in humans being the most preferred. The method may be used to close any type of wound, particularly wounds caused by trauma, surgery, disease, etc. The method is well suited to closing wounds in soft tissue or sealing soft tissue to a hard tissue. For example, suitable soft tissues that may welded include structures of the body that connect, envelope, support and/or move the structures of the body including muscle, tendons, ligaments, synovial tissue, fascia and other structures such as skin, mucosal membranes, organs, nerves, blood vessels and fatty tissue. Preferred soft tissue wounds treated with the instant method include those of the oral cavity such as the gingival tissue and periodontal pockets. Hard tissues include bone, tooth dentine and cartilage.

It will be further appreciated that functions or structures of a plurality of components or steps may be combined into a single component or step, or the functions or structures of one-step or component may be split among plural steps or components. The present invention contemplates all of these combinations. Unless stated otherwise, dimensions and geometries of the various structures depicted herein are not intended to be restrictive of the invention, and other dimensions or geometries are possible. Plural structural components or steps can be provided by a single integrated structure or step. Alternatively, a single integrated structure or step might be divided into separate plural components or steps. In addition, while a feature of the present invention may have been described in the context of only one of the illustrated embodiments, such feature may be combined with one or more other features of other embodiments, for any given application. It will also be appreciated from the above that the fabrication of the unique structures herein and the operation thereof also constitute methods in accordance with the present invention.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes.

What is claimed is:

1. A method of treating a wound, comprising:
disinfecting a wound by illuminating a photosensitizer composition in the wound; and
welding the wound closed by illuminating the wound.

2. The method of claim 1 wherein the welding step occurs substantially immediately after the disinfecting step.

3. The method of claim 1 wherein the photosensitizer composition comprises a solder composition.

4. The method of claim 1 claims wherein the welding step results in permanently closing the wound through a tissue welding process.

5. The method of claim 1 wherein the disinfecting step further comprises applying the photosensitizer composition, comprising an antimicrobial photosensitizer, to the wound.

6. The method of claim 1 wherein welding step further comprises bringing the edges of the wound together before illumination.

7. The method of claim 1 claims wherein the illuminating steps comprise illuminating with a laser, LED, or broadband light source.

8. The method of claim 1 wherein the photosensitizer composition comprises tetrapyrroles or derivatives thereof such as porphyrins, chlorins, bacteriochlorins, phthalocyanines, naphthalocyanines, texaphyrins, verdins, purpurins or pheophorbides, phenothiazines, or combinations thereof.

9. The method of claim 1 wherein the photosensitizer composition comprises an antimicrobial photosensitizer comprising methylene blue, indocyanine green or combinations thereof.

10. The method of claim 1 wherein the closing step comprising mechanically bringing edges of the wound together so that the photosensitizer composition and wound edges are in contact with each other.

11. The method of claim 1 wherein the photosensitizer composition is a liquid that gels upon application to the wound or a solid that liquefies upon application to the wound.

12. The method of claim 1 further comprising preparing the wound before applying the antimicrobial photosensitizer including optionally debriding the wound, removing foreign material, removing blood, controlling bleeding and/or drying the wound.

13. The method of claim 1 wherein the preparing the wound step comprises a light mediated method of controlling bleeding in the wound.

14. The method of claim 1 further comprising applying a solder composition to the wound before closing the wound.

15. The method of claim 14 claims wherein the photosensitizer composition comprises the solder composition.

16. The method of claim 14 wherein the solder composition comprises a proteinaceous compound, a lipid or combinations thereof.

17. The method of claim 14 wherein the photosensitizer composition comprises an antimicrobial photosensitizer, a proteinaceous compound, and a tissue penetration enhancer.

18. The method of claim 14, wherein the solder composition further comprises a therapeutic agent.

19. A method of treating a wound, consisting essentially:
applying a photosensitizer composition comprising an antimicrobial photosensitizer to the wound;
disinfecting a wound by illuminating the photosensitizer composition in the wound with a laser;
closing the wound; and
welding the wound closed by illuminating the wound with a laser.

20. The method of claim 19 wherein the closing and welding steps occur substantially immediately after the disinfecting step.

21. The method of claim 19 wherein the photosensitizer composition further comprises a solder composition comprising a proteinaceous compound.

22. The method of claim 19 further comprising preparing the wound before applying the photosensitizer composition including optionally debriding the wound, removing foreign material, removing blood, controlling bleeding and/or drying the wound.
